(19) United States
(12) Patent Application Publication
(10) Pub. No.: US 2017/0189326 A1
(43) Pub. Date: Jul. 6, 2017

(54) Topical Antiaging Polyphenol Compositions

(71) Applicants: Nicholas V. Perricone, Meriden, CT (US); Nancy Leung, Oakland, CA (US); Nicole Erickson, Walnut Creek, CA (US); Martin S. Flacks, San Francisco, CA (US)

(72) Inventors: Nicholas V. Perricone, Meriden, CT (US); Nancy Leung, Oakland, CA (US); Nicole Erickson, Walnut Creek, CA (US); Martin S. Flacks, San Francisco, CA (US)

(21) Appl. No.: 15/395,987

(22) Filed: Dec. 30, 2016

Related U.S. Application Data
(60) Provisional application No. 62/273,650, filed on Dec. 31, 2015.

(51) Int. Cl. A61K 8/97; A61K 8/46; A61K 8/35; A61Q 19/08; A61K 8/49

(52) U.S. Cl. CPC A61K 8/97; A61Q 19/08; A61K 8/498; A61K 8/35; A61K 8/46; A61K 2800/592

(57) ABSTRACT

Topical compositions and methods of use of topical compositions including a four component system consisting of olive polyphenols, turmeric curcuminoids, cruciferous sulforaphane and green tea catechins to improve the appearance of aging skin and to prevent and treat skin aging. The compositions are applied to aging skin to improve the appearance of aging skin, including surface spots, brown spots, red areas, wrinkles and texture and other artifacts of aging skin, as well as conditions of skin dryness, dullness, loss of elasticity, lack of radiance, exaggerated lines and wrinkles, spider vessels or red blotchiness.

### FIELD OF THE INVENTION

The present invention relates to topical compositions containing olive polyphenols, turmeric curcuminoids, cruciferous sulforaphane and green tea catechins in a dermatologically acceptable carrier and methods of use thereof. Application of these compositions to skin improves the appearance of aging skin and prevents and treats skin aging. More specifically, the present invention relates to methods of preventing the appearance of wrinkles and fine lines, dryness, dullness or lack of radiance of skin of the face neck and décolletage, and for treating the appearance of exaggerated lines and wrinkles, sagging, discoloration, or redness and blotchiness of such skin by application of compositions containing olive polyphenols, turmeric curcuminoids, cruciferous sulforaphane, and green tea catechins in a dermatologically acceptable carrier.

### BACKGROUND OF THE INVENTION

Human skin is constantly directly exposed to the air, solar radiation, environmental pollutants, or other mechanical and chemical insults, which are capable of inducing the generation of free radicals as well as reactive oxygen species (ROS) of our own metabolism. Extrinsic skin damage develops due to several factors: ionizing radiation, severe physical and psychological stress, alcohol intake, poor nutrition, overeating, environmental pollution, and exposure to UV radiation (UVR). It is estimated that among all these environmental factors, UVR contributes up to 80%. UV-induced generation of ROS in the skin develops oxidative stress, when their formation exceeds the antioxidant defiance ability of the target cell. The primary mechanism by which UVR initiates molecular responses in human skin is via photochemical generation of ROS, mainly formation of superoxide anion (O(2) (−) (.), hydrogen peroxide (H(2)O(2)), hydroxyl radical (OH(.)), and singlet oxygen ((1)O(2)). Over time, the presence of ROS will cause conditions of aging skin.

Moreover, when skin is inflamed from UVR, irritants, trauma, and other reasons, phospholipase-A-2 produces arachidonic acid from the phospholipid-rich membranes of the cell, resulting in the production of metabolites. Stabilization of the cell membrane can inhibit the inflammatory cascade, therefore preventing the inflammatory response. Arachidonic acid has a direct toxic effect on the mitochondria, resulting in the uncoupling of oxidative phosphorylation, resulting in free radical damage to the mitochondrial membrane.

Nuclear factor (erythroid-derived 2)-like 2 (“Nrf2”) is a transcription factor and basic leucine zipper protein that regulates the expression of antioxidant proteins that protect against oxidative damage triggered by injury and inflammation. Under normal or unstressed conditions, Nrf2 is kept in the cytoplasm by a cluster of proteins that degrade it quickly. Under oxidative stress, Nrf2 is not degraded, but instead travels to the nucleus where it binds to a DNA promoter and initiates transcription of antioxidative genes and their protein (AREs). Activation of Nrf2 results in the induction of many cytoprotective proteins and can reduce or prevent the formation of ROS. Current information indicates that Nrf2 and nuclear factor-kappa B (NFKB) pathways share common effectors and regulatory points and that the NFKB pathway is inhibited by several Nrf2 activators.

Polyphenols are efficacious in the treatment of skin damage because they are fat-soluble and readily disperse in cell membranes and other cellular components. Polyphenols readily penetrate skin. They are also active antioxidants that have been shown to scavenge superoxide radicals and inhibit neutrophilic respiratory bursts. Polyphenols act as free radical scavengers and neutralizers, and prevent the cross-linking of cell membranes that is often seen in its post-inflammatory phases. By the same token, polyphenol modulation of free radicals and other oxidative species appears to affect gene expression, including expression of nuclear factor K-B (NFKB), nitric oxide synthetase and other mediators at all stages of proinflammation and inflammation.

U.S. Pat. No. 6,437,004 discloses the use of olive oil polyphenol compositions to treat damaged skin tissue, particularly various types of dermatitis, rosacea, seborrhea, eczema, xerosis, psoriasis, thermal radiation and burns, and other types of inflammation. The compositions comprise about 0.1% to about 10% by weight of olive oil polyphenols consisting of hydroxytyrosol, oleoeuropeine, and mixtures thereof. Polyphenolic alteration of lipid peroxidation, protein cross-linking, growth factor stimulation, and membrane permeability may explain its negative effect on the symptoms of inflamed and aging skin.

Green tea benefits from a long term botanical tradition in Asia due to is preventative and protective effects on health. The cardinal antioxidant ingredient in green tea extract is green tea catechins, which comprise five major epicatechin derivatives: epicatechin, epigallocatechin, epicatechin gallate, gallocatechin gallate, and epigallocatechin gallate. Epigallocatechin gallate (EGCG) is one of green tea's most active polyphenols. It is well known to have powerful free radical scavenging and possess anti-inflammatory action on the skin. It also possess an inhibitory action on collagenase, thereby preventing wrinkle formation. However, most green tea extracts oxidize easily and are therefore difficult to formulate into stable topical compositions. Oxidation can cause unpleasant odors and discoloration in topical compositions, such as lotions and creams, which are commonly used for over the counter anti-aging treatments.

Curcumin is a compound isolated from turmeric. Its use is known for the chemoprevention and treatment of various skin diseases like scleroderma, psoriasis and skin cancer. Curcumin protects skin by quenching free radicals and reducing inflammation through NFKB inhibition.

Structurally, curcumin has two α,β unsaturated carbonyl groups and can hence act as a Michael reaction acceptor, thereby causing thiol modification of Keap-1. Studies on rats have shown, consistent with this notion, that oral administration of tetrahydrocurcumin (THC), which lacks an electrophilic α,β unsaturated carbonyl functional moiety, failed to induce NFr2-ARE binding as well as HO-1 induction. Farombi et al. 2008, Curcumin attenuate dimethylnitrosamine-induced liver injury in rats through nrf2-mediated induction of heme oxygenase-1, Food Chem Toxicol 46, 1279-1287. In contrast, oral administration of curcumin protected against dimethylnitrosamine-induced hepatic injury. More recent studies, have shown, however, that THC mediates its effects by activation of Nrf2 and its regulated enzymes glutamylcysteine ligase catalytic subunit and quinone oxidoreducatese1. Somparn et al., Protective Effects of Tetrahydrocurcumin and Curcumin against Doxorubicin and Cadmium-Induced Cytotoxicity in Chang Liver Cells, Trop J Pharm Res, May 2015; 14(5):769-776.

A major drawback of using curcumin in skin care products is it yellow coloring. WO 2007/02564 to Bommarito discloses incorporation of turmeric and its active ingredients into topical skin care foams, creams, lotions, ointments and sprayable solutions without turning the skin yellow. Such effect is achieved by combination with various berries, citrus juice, coffee beans, green tea extract or pomegranate extract.

Sulforaphane is an isothiocyanate that has been found to elevate cytoprotective enzymes and reduce skin sensitivity to erythema and protect against UVR-induced inflammation and edema. It is obtained from cruciferous vegetables, such as broccoli, brussel sprouts, cabbage cauliflower, bok choy, kale collards, watercress, kohlrabi, mustard, turnip, radish and arugula. Sulforphane is known to stimulate Nrf2 by disrupting the Nrf2/Keap 1 complex, thus activating the cellular detoxification system. However, many sulforaphanes have poor smell, low stability, and are not suitable for formulation into commercial topical compositions for treatment of skin.

U.S. 2014/0193480 to McWherter et al. discloses pharmaceutical compositions and topical formulations to treat inflammation, inhibit aberrant tissue renin-angiotensin factors (tRAS) and stabilize the extracellular matrix (ECM). The compositions comprise at least one active ingredient from five primary nutraceutical groups: (1) catechins, (2) curcuminoids, (3) isothiocyanates, (4) stilbenoids and (e) therapeutic essential oils. Topical compositions are disclosed to treat breast tissue, and as a “sports cream” or “muscle ache relief” cream.

It would be desirable to have improved polyphenol compositions and methods of treating and preventing the appearance of wrinkles and fine lines, dryness, dullness or lack of radiance of skin of the face neck and décolletage, and for treating the appearance of exaggerated lines and wrinkles, sagging, discoloration, or redness and blotchiness of such skin. In particular, it would be desirable to have methods of treating and preventing the appearance of wrinkles and fine lines, dryness, dullness or lack of radiance of skin of the face neck and décolletage, and for treating the appearance of exaggerated lines and wrinkles, sagging, discoloration, or redness and blotchiness of such skin involving the use of phytochemicals, such as naturally occurring polyphenols. It would be desirable to have topical compositions containing multiple polyphenol ingredients that have the potentials to activate Nrf2, such as olive polyphenol, turmeric curcuminoid, cruciferous sulforaphane and green tea catechins, that are stable in that they do not discolor and have a desirable smell after prolonged use, and can be commercially purchased without a prescription.

### SUMMARY OF THE INVENTION

It is an object of the invention to provide new and improved methods of treating and preventing the appearance of wrinkles and fine lines, dryness, dullness or lack of radiance of skin of the face neck and décolletage, and for treating the appearance of exaggerated lines and wrinkles, sagging, discoloration, or redness and blotchiness of such skin.

These and other objectives of the invention are accomplished by the present invention, which provides methods and dermatological compositions comprising a complex, or four component system, consisting of free radical-scavenging olive polyphenols, turmeric curcuminoids, cruciferous sulforaphane and green tea catechins, which when topically applied to exposed or affected skin areas can activate Nrf2 to treat and prevent the appearance of wrinkles and fine lines, dryness, dullness or lack of radiance, exaggerated lines and wrinkles, sagging, discoloration, and/or redness and blotchiness of skin. The amount of olive oil polyphenols, turmeric curcuminoids, cruciferous sulforaphane and green tea catechins used is not fixed per se, and necessarily dependent upon the concentration and type of each ingredient in the dermatological compositions, the user's skin type, and the severity, extent, and nature of the dermatological problem treated. In some typical embodiments, a composition comprising from about 0.01% to about 1.0% by weight olive polyphenol, 0.001% to about 0.05% by weight green tea catechins, 0.01% to about 2.0% by weight turmeric catechins, namely THC, and 0.1% to about 3.0% by weight cruciferous sulforaphane is applied to skin of the face neck and décolletage. In certain typical embodiments, 20% to 100% by weight of an Nrf2 activating complex consisting of olive oil polyphenols, turmeric curcuminoids, cruciferous sulforaphane and green tea catechins is formulated in a dermatologically acceptable carrier.

In some embodiments, the Nrf2 activating topical compositions applied to skin further comprise one or more adjunct active ingredients selected from the group consisting of fatty acid, fatty acid ester of ascorbic acid, and the mixture thereof. In some embodiments, the composition further comprises at least one adjunct active ingredient selected from the group consisting of tocotrienols, tocotrienol derivatives, vitamin E compositions enriched with tocotrienols or tocotrienol derivatives, vitamin C and vitamin C derivatives, and mixtures thereof. In certain preferred embodiments, the compositions comprise dimethyl MEA and or acetyl tyrosine. The adjunct active ingredients may be present from about 0.01% to about 20.0% by weight.

In some embodiments, the Nrf2 activating composition is a fluid or liquid solution. In certain embodiments, the composition is an oil in water emulsion and/or suspension. In some of these embodiments, the emulsifier comprises fatty acid derivatives of stearic acid. In other embodiments, the composition is a cream.

### DETAILED DESCRIPTION OF THE INVENTION

Aging skin is characterized histologically by cross-linking of collagen and elastin in the dermis. This results in loss of support seen clinically as sagging and wrinkling. The present invention recognizes these processes and provides methods to minimize both prospective and existing aging conditions. In particular, the present invention provides methods of applying topical compositions comprising a four component system consisting of olive polyphenols, turmeric curcuminoids, cruciferous sulforaphane and green tea catechins, such that the rate of regeneration of skin cell tissues is predominant over the rate of degeneration, thereby preventing skin aging conditions. It is believed that the inventive methods achieve such results by way of a synergistic combination of certain olive polyphenols, turmeric curcuminoids, cruciferous sulforaphane and green tea catechins, which when formulated in combination, activate Nrf2. It is believed that the combination of the four component system of the invention provides a synergistic effect that is not exhibited by use of olive polyphenols, turmeric curcuminoids, cruciferous sulforaphane and green tea catechins alone or in combination with other ingredients.

The term “topical composition” as used herein shall mean the complete product including olive polyphenols, turmeric curcuminoids, cruciferous sulforaphane and green tea catechins, carrier, and any adjuvants, thickeners, excipients, etc. as described herein which is applied to a person's skin.

The term “skin” means the keratinous surfaces skin, hair and nails. The term “skin” when used herein is in the broad sense meaning the skin of the face, body, and neck as well as the lips. Particularly preferred embodiments involve treatment and application of topical compositions the skin of the face neck and décolletage.

By “polyphenol” is meant any polyphenol that scavenges free radicals and exhibits antioxidant activity. Polyphenols are part of the so-called “polar fraction” of virgin olive oil, which is usually obtained by extraction with methanol: water systems (Tsimidou, M., et al., Food Chemistry 45: 141-144 (1992)). Many of these phenol fraction preparations contain other o-diphenols such as protocatechuic acid, caffeic acid, and syringic acid as well as hydroxytyrosol (Papadopoulos, G., and Boskou, D., J. Amer. Oil Chem. Soc. 68: 669-671 (1991)).

“Olive polyphenols” include the natural and synthetic compounds discussed in U.S. Pat. No. 6,437,004, incorporated herein by reference. Preferred olive polyphenols used in the inventive methods include olea europaea leaf extract, cetearyl olivate, sorbitan olivate, and squalene. A particularly preferred olive polyphenol is oleuropein.

A disadvantage of certain olive polyphenols is that they are very dark brown in color and difficult to formulate into commercially viable topical cosmetic compositions. Even those that are lighter in color may turn browner when combined with other ingredients. It is an advantage of the invention that olive polyphenols are able to be formulated into various stable topical presentations that do not discolor or produce unpleasant odor over the recommended shelf life. In particular, the inventors were able to formulate a commercially viable liquid solution, suspension, oil, tinted spf, mask and oil in water emulsions (moisturizer and eye cream).

In certain preferred embodiments, the olive polyphenol consists of C20+Cobiolive commercially available from Cobiosa. C20+ is characterized by its high content of hydroxytyrosol (over 20%), tyrosol and other polyphenols. In some embodiments, the olive polyphenol consists of Sabinsa's Oleuropein 80%, a standardized natural extract from Olea europaea (Olive) leaves that contains a minimum of 80% Oleuropein. A particularly preferred olive polyphenol for practicing the present invention is olea europaea leaf extract, available commercially as Eurol®BT, a natural blend of diphenolic compounds, including oleuropein.

Olive polyphenol may be present in topical compositions for use in inventive methods at 0.005% to about 2.0% by weight, more preferably 0.01 to 1.0% by weight, most preferably 0.1 to 0.5% by weight.

In preferred embodiments, the inventive topical compositions include from about 0.005% to about 0.5% by weight olea europaea leaf extract. In certain preferred embodiments, the inventive compositions comprise about 0.50% by weight of a blend of diphenolic compounds extracted from olea europaea leaf. In other preferred embodiments, the compositions of the invention comprise about 0.025% by weight of a blend of diphenolic compounds extracted from olea europaea leaf. In yet other preferred embodiments, the compositions comprise 0.05% by weight of a blend of diphenolic compounds extracted from olea europaea leaf.

“Green tea catechins” are a type of polyphenol obtained from the extracts of green tea. A preferred green tea catechin for use in the invention is epigallocatechin gallate (EGCG) and its derivatives, such as epigallocatechin gallatyl glucoside. The green tea catechins may be present in topical compositions for use in the invention at 0.001% to about 0.1% by weight preferable 0.001 to 0.05% by weight of a topical composition.

A particularly preferred source of EGCG available in a commercially water soluble form is Inoveol® by Induchem. As noted, most green tea extracts oxidize easily and are therefore difficult to formulate into stable topical compositions, especially lotions and creams that are not in air tight containers. These types of presentations are often preferred for anti-aging treatments. EGCG is a highly purified and stabilized form of green tea catechins. In combination with the olive polyphenol, turmeric curcuminoids, and cruciferous sulforaphane of the present invention this particular green tea extract was able to be formulated into various stable presentations that did not discolor or produce unpleasant odor over the recommended shelf life. In particular, the inventors were able to formulate a commercially viable liquid solution, suspension, oil, tinted spf, mask and oil in water emulsions (moisturizer and eye cream).

In preferred embodiments, the inventive topical compositions include from about 0.001% to about 0.01% by weight ECGC. In certain preferred embodiments, the inventive compositions comprise about 0.05% by weight ECGC. In other preferred embodiments, the compositions of the invention comprise 0.015% by weight ECGC. In yet other preferred embodiments, the compositions comprise 0.02%, 0.010%, or 0.005% by weight of ECGC.“Turmeric curcuminoids” refers to natural and synthetic sources of curcuminoids compounds that may be derived from turmeric, their prodrugs and conjugates, as well as formulations, liposomes and nanoparticles containing such compounds. Curcuma plant materials contain three different curcuminoid compounds including, as the predominant coloring compound, curcumin having a strong yellow color, and minor yellow and brownish-red compounds. The term “curcuminoids” includes curcumin (C), reddish orange and with two methoxy groups; demethoxy curcumin (DMC), orange-yellow with one methoxy group and bis-demethoxy curcumin (BDMC), yellow and without a methoxy group. As well the term turmeric curcuminoids includes hydrogenated derivatives of metabolism of such compounds, such as tetrahydrocurcumin (THC), hexahydro\curcumin (HHC) and octahydrocurcumin (OHC). In certain embodiments, the invention utilizes a combination of tetrahydrodemethoxydiferuloylmethane, tetrahydrobismethoxydiferuloylmethane, tetrahydrodiferuloylmethane, or combinations thereof.

Preferably, the invention utilizes THC, which is present in Curcumin C3 Reduct® sold by Sabinsa. An advantage of THC for topical uses is that it is colorless, and does not contain the staining yellow to brown pigment that many other curcuminoids possess. In some preferred embodiments, the invention utilizes Sabinsa SabiWhite tetracurcuminoids, a powdered extract that is also colorless.

Turmeric curcuminoids may be present in topical compositions for use in the inventive methods at about and 0.01% to about 2.0% by weight, preferably 0.5 to 2.0% by weight. In particularly preferred embodiments, THC GC tetracurcuminoids are formulated into topical compositions at about 0.5% to about 2% by weight of the composition.

In certain preferred embodiments, the inventive compositions comprise about 0.3% to about 0.5% by weight THC GC. In other preferred embodiments, the compositions of the invention comprise about 0.01% by weight THC GC. In yet other preferred embodiments, the composition comprises 0.1% to 0.2% by weight of THC GC. In combination with the olive polyphenol, green tea extracts, and cruciferous sulforaphane of the present invention, THC GC was able to be formulated into various stable presentations that did not discolor or produce unpleasant odor over the recommended shelf life. In particular, the inventors were able to formulate a commercially viable liquid solution, suspension, oil, tinted spf, mask and oil in water emulsions (moisturizer and eye cream) with pleasant odor and that did not discolor.

“Cruciferous sulforaphane” refers to the sulforaphane C₆H₁₁NOS₂ compound found and/or extracted from a broad range of vegetables of the Brassica oleracea genus. A feature of cruciferous plants is the synthesis of sulfur-rich compounds, such as glucosinolates. Glucosinolates are synthesized and stored in plants as relatively stable isothiocyanate precursors. Glucosinolates are soluble in water due to the glucose molecule, which imparts hydrophilic characteristics, unlike isothiocyanates which have hydrophobic properties. Glucosinolates are hydrolyzed when plant tissue is broken as a result of mechanical damage. When this occurs, thioglucosidase enzyme and myrosinase are brought into contact with the substrate and release glucose molecules, bisulfate and the corresponding aglycone. Subsequently, an intramolecular arrangement generates isothiocyanates, and other compounds.

Many sulforaphanes, such as broccoli sprout extracts, have poor smell and are difficult to formulate in topical compositions for over the counter use. Additionally, many commercially available sulforaphanes are provided with carriers, e.g., sunflower oil, that are not desirable for cosmetic skin care applications. It is an advantage of the invention that cruciferous sulforaphane can be formulated into various formulation types for topical cosmetic use that have pleasant smell and/or undetectable odor and free of sunflower oil and other like oil carriers.

Cruciferous sulforaphane may be present in topical compositions for use in the inventive methods at about 0.1% to about 4.0% by weight, preferably, 0.5 to 3% by weight of a topical compositions.

Preferably, the invention utilizes cress sprout sulforaphane (lepidium sativum sprout extract). Garden cress has a spicy aroma and a refreshing, peppery-pungent taste. Like the other members of the Brassicaceae family, garden cress owes its aroma to isothiocyanates. The sprouts of these vegetables contain the highest concentration of phytonutrients. The cosmetic ingredient Detoxophane, commercially available from Mibelle Biochemistry, is a particularly preferred form of garden cress sulforaphane obtained from hydroponic cultivation of 4 to 5 day old garden cress sprouts. The composition of the ingredient is the following (INCl): Lepidium Sativum Sprout Extract, Glycerin, Lecithin, Phenoxyethanol and Aqua. For a better skin uptake, the actives of Detoxophane are incorporated into liposomes. Encapsulation allows the compounds to be more stable and have low to no odor.

In certain preferred embodiments, the inventive compositions contain encapsulated cress sprout sulforaphane at around 0.5% to around 3% by weight. In other preferred embodiments, the compositions of the invention contain around 2.00% by weight encapsulated cress sprout sulforaphane. In yet other preferred embodiments, the composition comprises 0.1% to 0.8% by weight of encapsulated cress sprout sulforaphane. In combination with the olive polyphenol, green tea extracts, and turmeric curcuminoids of the present invention, encapsulated cress sprout sulforaphane was able to be formulated into various stable presentations that did not discolor or produce unpleasant odor over the recommended shelf life. In particular, the inventors were able to formulate a commercially viable liquid solution, suspension, oil, tinted spf, mask and oil in water emulsions (moisturizer and eye cream) with pleasant odor and that did not discolor and was free of sunflower oil.

In one aspect, the present invention provides application of topical compositions comprising olive polyphenols, turmeric curcuminoids, cruciferous sulforaphane, and green tea catechins to skin for improving the appearance of aging skin and/or treating or preventing skin aging. The compositions are expected to help address severe skin dryness, dullness, loss of elasticity, lack of radiance, exaggerated lines and wrinkles, spider vessels or red blotchiness. Particularly, marionette lines, smile lines, deep nasolabial fold lines, crow's feet, fine lines/wrinkles, vertical lines between the eyebrows, horizontal forehead lines, sagging thin/frail skin, skin redness and dullness may be improved using the compositions of the invention. When applied to skin, compositions of the present invention are expected to show improvement in surface spots, brown spots, red areas, wrinkles and texture and other artifacts of aging skin. The term “surface spots” refers to brown or red spots which include freckles, acne marks or scars, hyperpigmentation and vascular lesions. The term “brown spots” refers to those caused by an excess of melanin on and within the skin, these lesions include freckles, melasma, hyperpigmentation and lentigines. The term “red areas” refers to various skin conditions such as acne, rosacea, inflammation and spider veins that have apparent red structures due to the blood vessels and hemoglobin contained in the papillary dermis. The term “wrinkles” refers to fine lines, furrows, folds and creases in the skin. Wrinkles are associated with decreased skin elasticity. The term “texture” refers to gradations in the skin's color and tone and surface peaks and valleys that are analyzed to measure smoothness.

After treatment for the recommended period of time, e.g. once to twice daily for a minimum of 2-4 weeks, it is expected that decreased inflammation, irritation, and erythema of the skin will be observed, along with an increased skin elasticity and suppleness. Particularly, marionette lines, smile lines, deep nasolabial fold lines, crow's feet, fine lines/wrinkles, vertical lines between the eyebrows, horizontal forehead lines, sagging thin/frail skin, skin redness and dullness are reduced. The present invention thus is expected to improve the appearance of skin, prevent and treat skin aging, dryness, dullness, loss of elasticity and lack of radiance. Particularly, treatments may be used to prevent or retard the appearance of spider vessels or red blotchiness associated with menopausal skin. In another embodiment, treatments may be used to prevent or treat exaggerated lines and wrinkles.

Only effective amounts of topical compositions containing olive polyphenols, turmeric curcuminoids, cruciferous sulforaphane and green tea catechins are needed to achieve the aforementioned benefits and prevent typical aging effects on the skin. Generally, topical application to skin tissue is accomplished in association with a dermatologically acceptable carrier, and particularly one in which the olive polyphenols, turmeric curcuminoids, cruciferous sulforaphane and green tea catechins are soluble per se or is effectively solubilized (e.g., as a suspension, emulsion or microemulsion). Where employed, the carrier is inert in the sense of not bringing about a deactivation or oxidation of the glutathione derived active ingredient(s), and in the sense of not bringing about any adverse effect on the skin areas to which it is applied.

In particularly preferred methods a complex consisting of oleuropein, EGCG, tetrahydrocurcuminoids, and cress sprout sulforaphane is formulated into various topical formulations at 10% to 100% by weight. For example, the complex is present in a serum at 90% to 100% by weight, in various creams and moisturizers at 20% to 40% by weight and in a mask treatment at 5% to 10% by weight.

In one preferred practice of the invention, the complex of olive polyphenols, turmeric curcuminoids, cruciferous sulforaphane and green tea catechins is applied in admixture with a dermatologically acceptable carrier or vehicle (e.g., as a fluid, lotion, cream, ointment, serum or the like) so as to facilitate topical application and, in some cases, provide additional therapeutic effects as might be brought about, e.g., by moisturizing of the affected skin areas. While the carrier for the topical composition can consist of a relatively simple solvent or dispersant such as water, it is generally preferred that the carrier comprise a composition more conducive to topical application, and particularly one which will form a film or layer on the skin to which it is applied so as to localize the application and provide some resistance to washing off by immersion in water or by perspiration and/or aid in the percutaneous delivery of the active agent(s). Many preparations are known in the art, and include lotions containing oils and/or alcohols and emollients vegetable oils, hydrocarbon oils and waxes, silicone oils, animal or marine fats or oils, glyceride derivatives, fatty acids or fatty acid esters, or alcohols or alcohol ethers, lecithin, lanolin and derivatives, polyhydric alcohols or esters, wax esters, sterols, phospholipids and the like, and generally also emulsifiers (nonionic, cationic or anionic), although some of the emollients inherently possess emulsifying properties. In certain preferred embodiments, the carrier is an oil in water emulsion. In other preferred embodiments the carrier is an alcohol solvent system.

Generally in the practice of methods of the invention, the topical composition is topically applied to the skin areas, such as that of the face, at predetermined intervals often as a moisturizer, lotion, or cream, it generally being the case that gradual improvement is noted with each successive application. Although immediate effects can be observed, enhanced results are observed when the topical composition is applied twice daily, preferably in the morning and evening. Insofar as has been determined based upon clinical studies to date, no adverse side effects are encountered. It is an advantage of the invention that compositions of the invention do not require a pharmaceutical prescription.

Some embodiments of this invention contain at least one other adjunct active ingredient in addition to olive polyphenols, turmeric curcuminoids, cruciferous sulforaphane and green tea catechins. Adjunct ingredients may be present in an amount ranging from 0.01% to about 20% by weight of the composition. They include, but are not limited to one or more of: caffeine, vitamin D3, lipoic acid; α-hydroxy acids such as glycolic acid or lactic acid; ascorbic acid and its derivatives, especially fatty acid esters of ascorbic acid; polyenylphosphatidylcholine; or tocotrienols and tocotrienol derivatives and vitamin E compositions enriched with tocotrienols or tocotrienol derivatives; and neuropeptides. Preferred adjunct agents include caffeine, tocopherol, tocopherol acetate, acetyl tyrosine, palmitoyl tripeptide-5, thioctic acid, hexylene glycol, magnesium ascorbyl phosphate and tetrahexyldecyl ascorbate.

The topical composition of the invention can contain additional ingredients commonly found in skin care compositions and cosmetics, such as, for example, tinting agents, emollients, skin conditioning agents, emulsifying agents, humectants, preservatives, antioxidants, perfumes, chelating agents, etc., provided that they are physically and chemically compatible with other components of the composition.

Preservatives include, but are not limited to, C₁-O₃ alkyl parabens and phenoxyethanol, typically present in an amount ranging from about 0.1% to about 2.0% by weight percent, based on the total composition. Preferred preservatives include phenoxyethanol and/or sodium benzoate.

Emollients, typically present in amounts ranging from about 0.01% to 10% of the total composition include, but are not limited to, fatty esters, fatty alcohols, mineral oils, polyether siloxane copolymers, docosahexanoic acid (DHA) and mixtures thereof. Preferred emollients include isohexadecane, cetyl alcohol, cetearyl alcohol, stearyl alcohol, ethylhexyl palmitate, and avocado oil.

Humectants, typically present in amounts ranging from about 0.1% to about 5% by weight of the total composition include, but are not limited to, polyhydric alcohols such as glycerol, polyalkylene glycols (e.g., butylene glycol, propylene glycol, dipropylene glycol, polypropylene glycol, and polyethylene glycol) and derivatives thereof, alkylene polyols and their derivatives, sorbitol, hydroxy sorbitol, hexylene glycol, 1,3-dibutylene glycol, 1,2,6-hexanetriol, ethoxylated glycerol, propoxylated glycerol, and mixtures thereof. Preferred humectants are glycerin and glycol derivatives, such as hexylene glycol, ethylhexylglycerin, glyceryl stearate, peg-12 glyceryl dimyristate, and caprylyl glycol and shea butter.

Emulsifiers, typically present in amounts from about 1% to about 15% by weight of the composition, include, but are not limited to, polysorbate 20, stearic acid, cetyl alcohol, stearyl alcohol, steareth 2, steareth 20, acrylates/C10-30 alkyl acrylate crosspolymers, silicones, dimethylethanolamine (DMAE), phosphatidylcholine (PPC), docosahexanoic acid (DHA) and mixtures thereof. Preferred emulsifiers are polysorbate 20 and dimethylaminoethanol, also known as DMAE.

Another preferred emulsifier is PPC. By “polyenylphosphatidylcholine (PPC)” it meant any phosphatidylcholine (PC) bearing two fatty acid substituents, wherein at least one is an unsaturated fatty acid with at least two double bonds. Preferred polyenylphosphatidylcholines contain at least one linoleic (18:2) group, most preferably two, in a cis geometrical configuration typical of natural products, which presents in the preparation at levels of at least about 25%, preferably at least about 40% by weight. Other forms of PPC can also be used as those set out in U.S. Pat. No. 6,797,459 at column 3 lines 34 to 52. PPC itself is an active antioxidant that has been shown to protect against lipid peroxidation and liver damage, including fibrosis and cirrhosis (Aleynik, S. I., et al., J. Investig. Med. 47: 507-512 (1999)). Moreover, because PC itself is a major constituent of cell membranes, PPC greatly enhances the antioxidant activity of the composition because it facilitates the niacinamide nucleotide to penetrate and disperse in cell membranes in quantities sufficient to reach therapeutic levels.

Chelating agents, typically present in amounts ranging from about 0.01% to about 2% by weight, include, but are not limited to, ethylenediamine tetraacetic acid (EDTA) and derivatives and salts thereof, dihydroxyethyl glycine, tartaric acid, and mixtures thereof.

Antioxidants, typically present in an amount ranging from about 0.01% to about 0.75% by weight of the composition, include, but are not limited to, butylated hydroxy toluene (BHT); vitamin C and/or vitamin C derivatives, such as fatty acid esters of ascorbic acid, particularly ascorbyl palmitate; butylated hydroanisole (BHA); phenyl-α-naphthylamine; hydroquinone; propyl gallate; nordihydroquiaretic acid; vitamin E and/or derivatives of vitamin E, including tocotrienol and/or tocotrienol derivatives; calcium pantothenates; and mixtures of any of these. Particularly preferred antioxidants are those that provide additional benefits to the skin, such as ascorbyl palmitate, alpha-lipoic acid, magnesium ascorbyl phosphate and tetrahexyldecyl ascorbate.

In certain preferred embodiments, in which the carrier for the Nrf2 complex is a fluid or serum, bilberry fruit extract, sugar maple extract, and/or sugar cane extract may be included in the compositions as antioxidant and exfoliating agents.

Perfumes and fragrances include citrus aurantium dulcis (orange) fruit extract, citrus limon (lemon) fruit extract and the like.

Buffering agents are employed in many compositions. Preferably, the amount of buffering agent is one that results in compositions having a pH ranging from about 4.0 to about 6.0, more preferably from about 4.5 to about 6.0, most preferably from about 4.5 to about 5.5. Typical buffering agents are chemically and physically stable agents commonly found in cosmetics, and can include compounds that are also adjunct ingredients such as citric acid, malic acid, and glycolic acid buffers.

Additional ingredients and methods as disclosed in U.S. Pat. Nos. 5,376,361; 5,409,693; 5,545,398; 5,554,647; 5,574,063; 5,643,586; 5,709,868; 5,879,690; 6,191,121; 6,296,861; 6,437,004; 6,979,459; 8,609,604; 8,609,618, 9,023,801; 9,029,317; and 9,034,926; which are hereby incorporated by reference, may also be used.

The methods of use involving application of a combination of olive polyphenols, turmeric curcuminoids, cruciferous sulforaphane and green tea catechins in a dermatologically acceptable carrier are expected to improve the appearance of aging skin, including surface spots, brown spots, red areas, wrinkles and texture and other artifacts of aging skin, as well as conditions of skin dryness, dullness, loss of elasticity, lack of radiance, exaggerated lines and wrinkles, spider vessels or red blotchiness. The appearance of marionette lines, smile lines, deep nasolabial fold lines, crow's feet, fine lines/wrinkles, vertical lines between the eyebrows, horizontal forehead lines, sagging thin/frail skin, skin redness and dullness may be improved using the methods of the invention.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention.

What is claimed is:

1. A method of topical use of compositions comprising a four component system consisting of olive polyphenol, turmeric curcuminoid, cruciferous sulforaphane and green tea catechin for improving the appearance of aging skin and to treat or prevent skin aging.

2. The method of claim 1, wherein the olive polyphenol is olea europaea leaf extracts.

3. The method of claim 1, wherein the green tea catechin is epigallocatechin gallate.

4. The method of claim 1, wherein the turmeric curcuminoid is tetrahydrocurcumin.

5. The method of claim 1, wherein the sulforaphane is cress sprout sulforaphane.

6. The method of claim 1, wherein the olive polyphenol comprises about 0.005% to about 2.0% by weight of the system.

7. The method of claim 1, wherein the turmeric curcuminoid comprises about 0.01% to about 2.0% by weight of the system.

8. The method of claim 1, wherein the cruciferous sulforaphane comprises about 0.1% to about 3.0% by weight of the system.

9. The method of claim 1, wherein the green tea catechin comprises about 0.001% to about 0.05% by weight of the system.

10. The method of claim 1, wherein the four component system is formulated into a dermatological carrier at about 10% to about 100% by weight.

11. A method of topical use of compositions comprising a four component system consisting of olea europaea leaf extracts, tetrahydrocurcumin, cress sprout sulforaphane, and epigallocatechin gallate.

12. The method of claim 11, wherein the system consists of about 0.50% by weight olea europaea leaf extracts, about 1.00% by weight tetrahydrocurcumin, about 2.00% by weight cress sprout sulforaphane, and about 0.05% by weight epigallocatechin gallate.

13. The method of claim 12, wherein the four component system is formulated into a dermatological carrier at about 10% to about 100% by weight.

14. A topical composition for application to aging skin, comprising
olive polyphenol,
turmeric curcuminoid,
cruciferous sulforaphane, and
green tea catechins in a dermatologically acceptable carrier.

15. The topical composition of claim 14, wherein
the olive polyphenol consists essentially of olea europaea leaf extracts;
the turmeric curcuminoid consists essentially of tetrahydrocurcumin;
the cruciferous sulforaphane consists essentially of cress sprout sulforaphane; and
the green tea catechins consists essentially of epigallocatechin gallate.

16. The composition of claim 15, comprising
about 0.05% to about 0.5% by weight olea europaea leaf extracts,
about 0.01% to about 1.00% by weight tetrahydrocurcumin,
about to about 2.00% by weight cress sprout sulforaphane, and
about 0.001% to about 0.05% by weight epigallocatechin gallate.

17. The composition of claim 15, comprising about 0.5% by weight olea europaea leaf extracts, about 1.00% by weight tetrahydrocurcumin, about 2.00% by weight cress sprout sulforaphane, and about 0.05% by weight epigallocatechin gallate.

18. The composition of claim 15, wherein the olea europaea leaf extracts, tetrahydrocurcumin, cress sprout sulforaphane, and epigallocatechin gallate are formulated into a mixture that comprises about 10% to about 100% of the topical composition.

19. The composition of claim 15, wherein the cress sprout sulforaphane is encapsulated.

20. The composition of claim 14, wherein the dermatologically acceptable carrier comprises lecithin and water.
